SEQUENCE LISTING (3 sequences)

SEQ ID NO: 1 - DNA, 720 bp, Bacillus subtilis
atggaaaaaccaaaatacaaacgtatcgtattaaagcttagcggggaagcattggcaggagaacagggaaatggaattaacccgactgtcattcaatccattgcaaagcaagtgaaggaaatcgctgagcttgaagtcgaagtggctgttgttgtaggcggcggcaactatggcgcggaaaaaacaggaagtgacctgggcatggaccgcgcgactgctgactatatgggaatgctggcgacagtaatgaattcgcttgctcttcaagacagcttggaaacactcggaatccagtccagagtgcaaacatccattgaaatgagacaagttgctgaaccgtacataagaagaaaagcgatacgccacttagagaaaaaacgtgtcgttattttcgctgcgggcacaggaaacccatatttctcaactgatacgacagctgcactgcgggctgctgaaattgaggcagacgttattttaatggctaaaaataacgttgacggtgtgtataatgctgatcctagaaaagatgaatcagctgttaagtatgaatcactttcttatcttgacgttcttaaagacggcctggaagtcatggattcaacagcttcctctttatgcatggacaatgacattccgcttatcgtcttttctattatggaagaaggaaatatcaaacgtgccgttatcggtgaatcaatcggaacgatcgtgagggggaaa

SEQ ID NO: 2 - DNA, 711 bp, Haemophilus influenzae
atgagccaaccaatttataaacgtattttattgaaattaagcggtgaagcattacaaggagaagatggtcttggtatcgatcctgcgattctcgatcgtatggctgttgaaattaaagaattagtggagatgggtgtggaagtcagtgtcgttctcggtggtggcaacttattccgtggcgcaaaactagcaaaagcggggatgaatcgcgtggtgggcgatcatatgggaatgcttgctactgtgatgaatggtttggcaatgcgtgattctttattccgtgctgatgtgaacgcaaaattaatgtccgctttccaattaaatggtatttgcgatacttataactggtctgaagctatcaaaatgttacgcgaaaaacgcgtagtcattttctctgcgggaacgggaaatccattctttaccactgattctaccgcttgtttgcgtggtattgaaattgaagctgatgttgtgttgaaagcgactaaagttgatggtgtgtatgattgtgatcctgcgaaaaatcctgatgcaaaactttataaaaatttaagttatgcagaagtgatcgataaagaattaaaagtgatggacttatcggcgtttactttagctcgcgatcatggcatgccgattagagtgttcaatatgggtaaacctggagcattacgtcaagtagtgactggtactgaagaaggcaccactatttgt

SEQ ID NO: 3 - DNA, 786 bp, Mycobacterium tuberculosis
atgacagagcccgatgtcgccggcgcgccggcttcgaagccggagcctgcgagcactggcgccgcgtcggcggctcagctgtccgggtactcgcgagtgttgctcaagctcggcggggaaatgttcggcggaggccaggtcgggctggatcccgatgtcgtcgcgcaggtggcccgccagatcgccgatgtggtgcgcggcggcgtccagatcgccgtcgtgatcggcggcggcaactttttccggggcgcacagctgcagcagctcggtatggagcgcaccaggtcggactatatgggaatgctcggcactgtcatgaacagccttgcactgcaagacttcctggagaaggaaggcatcgttacccgagtccagaccgcgatcaccatgggccaggtggccgaaccgtacctgccgttgcgggccgtccgccacctggagaagggacgggtggtgatcttcggtgccggcatggggctgccgtacttttcgacggataccaccgcagcgcagcgtgcgctggagatcggtgccgacgtggtcttgatggccaaagcggtcgacggtgtgtttgctgaggatccgcgggtgaaccccgaggccgaactgctcaccgccgtcagtcatcgtgaggtcctcgaccgcgggctgcgagtagccgacgccaccgcgttcagcctttgcatggacaatggcatgccgatcctggtgttcaacctgctgaccgatggcaatatcgcccgtgctgttcggggtgagaagatcggaacgctggtcaccacctaa

(19) United States
(12) Patent Application Publication
(10) Pub. No.: US 2002/0119506 A1
(43) Pub. Date: Aug. 29, 2002

(54) Genes encoding UMP kinase, methods for purifying UMP kinase and methods of characterizing UMP kinase

(75) Inventors: Octavian Barzu, Anthony (FR); Ann-Marie Gilles, Villebon/Yvette (FR); Hiroshi Sakamoto, Meudon (FR); Helene Munier-Lehmanni, Meudon (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: INSTITUT PASTEUR, Paris (FR)

(21) Appl. No.: 10/017,451

(22) Filed: Dec. 18, 2001

Related U.S. Application Data
(60) Provisional application No. 60/255,894, filed on Dec. 18, 2000.

(51) Int. Cl. C12Q 1/48; C12N 9/12; C07H 21/04; C12P 21/02; C12N 5/06

(52) U.S. Cl. 435/15; 435/194; 435/320.1; 435/325; 435/69.1; 536/23.2

(57) ABSTRACT

The present invention relates to the identification of the pyrH/smbA gene which encodes the UMP kinase gene in B. subtilis, M. tuberculosis, and H. influenzae, which are activated, stablilized an inhibited by specific nucleotides, methods of purifying UMP kinase, methods of characterizing UMP kinase and methods of stablizing UMP kinase.

### BACKGROUND OF THE INVENTION

The phosphorylation of UMP and CMP in eucaryotes is carried out by a single polypeptide. UMP/CMP kinases from Saccharomyces cerevisiae, Dictyostelium discoideum, Arabidopsis thaliana or pig muscle resemble adenylate kinase from muscle cytosol [Müller-Dieckmann, 1994 #717; Scheffzek, 1996 #750; Zhou, 1998 #1538; Okajima, 1995 #276; Dreusicke, 1988 #22]. Enteric bacteria contain separate UMP and CMP kinases and mutants defective in the gene encoding UMP kinase (pyrH/smbA) and CMP kinase (mssA/cmk) from E. coli or Salmonella typhimurium were isolated and characterized many years ago [Ingraham, 1972 #681; Beck, 1974 #636; Piérard, 1976 #735]. The recombinant UMP- and CMP kinases from E. coli were characterized in much detail [Serina, 1995 #95 1; Serina, 1996 #1058; Bucurenci, 1998 #985; Landais, 1999 #1080; Bucurenci, 1996 #984; Briozzo, 1998 #1651]. Thus, the bacterial UMP kinase is a homohexamer whose primary structure is divergent from that of other nucleoside monophosphate (NMP) kinases. The enzyme has an absolute specificity for UMP as substrate and is controlled allosterically by GTP (activator) and UTP (inhibitor) [Serina, 1995 #951]. CMP kinase from E. coli is a monomer which acts preferentially on CMP and dCMP [Bucurenci, 1996 #984]. Although the enzyme has little overall sequence identity with other known NMP kinases, it has in common with these enzymes a central parallel β-sheet, the strand of which are connected by α-helices. A property which is unique to the bacterial CMP kinase is a 40-residue insert situated within the CMP binding site and consisting of a three-stranded antiparallel β-sheet and two α-helices [Briozzo, 1998 #1651].

Attempts in the past to isolate a specific UMP kinase from B. subtilis failed. It was suggested that phosphorylation of UMP in this bacterium is accomplished by a CMP kinase with a broader specificity for pyrimidine nucleotides than the enzyme from E. coli [Waleh, 1976 #278]. The deleterious effect of disruption of cmk/jofC gene in B. subtilis [Sorokin, 1995 #277], was in line with this interpretation. Thanks to the genome sequencing programs, the pyrH gene was identified in all investigated bacteria, including B. subtilis. On the other hand, the pyrH gene from Lactococcus lactis, a bacterium similar to B. subtilis in the metabolism of pyrimidine nucleotides, complements a temperature sensitive pyrh mutation in E. coli demonstrating the ability of the encoded protein to synthesize UDP [Wadskov-Hansen, 2000 #1878].

These observations reopened the question of the role played by UMP kinase in the metabolism of B. subtilis and in gram positive organisms in general, and prompted us to clone the pyrH gene from B. subtilis and to examine the structural and catalytic properties of the recombinant protein. A striking characteristic of B. subtilis UMP kinase in comparison with the E. coli enzyme is its very low activity in the absence of GTP. On the other hand, the enzyme is unstable in the absence of UTP both in crude extract or under purified form. Antibodies against the recombinant UMP kinase identified the enzyme in the B. subtilis proteome, and immunoelectron microscopy confirmed the peripheral distribution of UMP kinase in this organism which extends our previous observations on E. coli enzyme.

In addition, we have successfully isolated and characterized clones of the pyrH/subA gene which encodes UMP kinase from M. tuberculosis and H. influenzae.

### SUMMARY OF THE INVENTION

Accordingly, an object of the present invention is to identify and characterize specific genes which encode UMP kinase. Characterisation includes determination of the sensitivity to nucleotides and nucleotide analogs. For example, activation in the presence of UTP and inhibition in the presence of GTP.

The pathway leading to synthesis of UMP in prokaryotes is also present in Bacillus subtilis, a gram positive bacterium whose whole genome sequence was reported three years ago. A question still waiting an answer was related to the existence of an active UMP kinase in this organism, as attemps to isolate the enzyme were unsuccessful. The gene encoding the UMP kinase (pyrH/smbA) is present in B. subtilis, and its open reading frame is transcribed in vivo into a functional enzyme of X aminoacid residues, (˜0.15% of total proteins of B. subtilis). UMP kinase from B. subtilis is extremely unstable in the absence of cofactors. UTP in millimolar concentrations increased significantly the stability of the protein in long-term storage either in bacterial extract or under purified form. The specific activity of the purified enzyme in the presence of GTP which acts as an activator is of 25 μmol/min⁻¹/mg of protein⁻¹. Taking into consideration, the specific activity of UMP kinase from E. coli under identical conditions 150 μmol/min⁻¹/mg of protein⁻¹ and the relative abundance of the enzyme 0.05% of total E. coli proteins it might be estimated that the rate of UMP phosphorylation in B. subtilis is approximately 50% of that in E. coli. In the absence of GTP the activity of B. subtilis UMP kinase is twenty times lower, indicating the major role of this nucleotide in controlling catalysis both in vitro and in vivo. Activation by GTP is specific: contrary to E. coli UMP kinase which is also activated by GMP, the B. subtilis enzyme is insensitive to the latter nucleotide. Only dGTP and GMP-PNP can activate at significant rates the enzyme from B. subtilis. UTP inhibits the UMP kinase from B. subtilis, with a lower affinity than that shown towards the E. coli UMP kinase. Antibodies directed against the recombinant enzyme demonstrated the peripheral distribution of UMP kinase in B. subtilis extending our previous observations on the enzyme from Escherichia coli.

### DETAILED DESCRIPTION OF THE INVENTION

The term “nucleic acid molecule” as used herein means RNA or DNA, including cDNA, single or double stranded, and linear or covalently closed molecules. A nucleic acid molecule may also be genomic DNA corresponding to the entire gene or a substantial portion thereof or to fragments and derivatives thereof. The nucleotide sequence may correspond to the naturally occurring nucleotide sequence or may contain single or multiple nucleotide substitutions, deletions and/or additions including fragments thereof. All such variations in the nucleic acid molecule retain the ability to encode a biologically active protein when expressed in the appropriate host or an enzymatically active fragment thereof. The nucleic acid molecule of the present invention may comprise solely the nucleotide sequence encoding a protein or may be part of a larger nucleic acid molecule that extends to the gene for the protein. The non-protein encoding sequences in a larger nucleic acid molecule may include vector, promoter, terminator, enhancer, replication, signal sequences, or non-coding regions of the gene.

Those nucleotide sequences which are substantially identical to those specifically disclosed are included in the present inveniton. Such sequences are those hybridize to each other under stringent conditions and encode UMP kinase. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH8 and a temperature of approximately 60° C. These methods and others known in the art are described in Sambrook et al, Molecular Cloning: A Laboratory Manual, Cold Spring Harbor Laboratory Press, Cold Spring Harbor, N.Y. (1989).

Nucleotide sequences are also substaintially identical for purposes of this application when the polypeptides which they encode are substantially identical. Thus, where one nucleic acid sequence encodes essentially the same polypeptide as a second nucleic acid sequence, the two nucleic acid sequences are substantially identical, even if they would not hybridize under stringent conditions due to silent substitutions permitted by the genetic code (see, Darnell et al. (1990) Molecular Cell Biology, Second Edition Scientific American Books W. H. Freeman and Company New York for codon degeneracy and the genetic code).

The conditions for culturing the microorganisms can be chosen from those which are preferable for their growth. Any natural- and synthetic-nutrient culture media can be used for culturing the microorganisms used in the present process as long as the microorganisms can grow therein and produce the present enzyme. The carbon sources used in the present invention are those which can be utilized by the microorganisms; for example, saccharides such as maltose, trehalose, dextrins, and starches, and natural substances which contain saccharides such as molasses and yeast extracts can be used. The concentration of these carbon sources contained in the culture media is chosen depending on their types. The nitrogen sources used in the present invention are, for example, inorganic nitrogen-containing compounds such as ammonium salts and nitrates, and organic nitrogen-containing compounds such as urea, corn steep liquor, casein, peptone, yeast extract, and meet extract. If necessary, inorganic compounds, for example, salts of calcium, magnesium, potassium, sodium, phosphoric acid, manganese, zinc, iron, copper, molybdenum, and cobalt can be used in the present invention.

After culturing the microorganisms, the present enzyme can be collected from the cultures. Because the enzyme activity may be generally present intracellularly, intact and processed cells can be obtained as crude enzymes. Whole cultures can be also used as crude enzymes. Conventional solid-liquid separation methods can be used to separate cells and nutrient culture media; for example, methods to directly centrifuge the cultures, those to filtrate the cultures after adding filer aids to the cultures or after pre-coating, and those to filter the cultures using membranes such as plain filters and hollow fibers can be used. The intact and processed cells per se can be used as crude enzymes, and if necessary, they can be prepared into partially purified enzymes.

The types of the processed cells include protein fractions of cells, immobilized substances of the intact and processed cells, and cells which were dried, lyophilized, and treated with surfactants, enzymes, ultrasonication, mechanical grinding, and mechanical pressure.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322. pBR322 contains genes for ampicillin and tetracycline resistance and thus provides simple means for identifying transformed cells. To construct an expression vector using pBR322, an appropriate promoter and a DNA sequence are inserted into the pBR322 vector.

Promoter sequences commonly used for recombinant prokaryotic host cell expression vectors include β-lactamase (penicillinase), lactose promoter system (Chang et al., Nature275:615, (1978); and Goeddel et al., Nature 281:544, (1979)), tryptophan (trp) promoter system (Goeddel et al., Nucl. Acids Res. 8:4057, (1980)), and tac promoter (Sambrook et al, Molecular Cloning: A Laboratory Manual, Cold Spring Harbor Laboratory, p. 412 (1982)).

The proteins of the present invention may, when beneficial, be expressed as a fusion protein that has the protein attached to a fusion segment. The fusion segment often aids in protein purification, e.g., by permitting the fusion protein to be isolated and purified by affinity chromatography. Fusion proteins can be produced by culturing a recombinant cell transformed with a fusion nucleic acid sequence that encodes a protein including the fusion segment attached to either the carboxyl and/or amino terminal end of the protein. Preferred fusion segments include, but are not limited to, glutathione-S-transferase, β-galactosidase, a poly-histidine segment capable of binding to a divalent metal ion, and maltose binding protein.

According to the present invention, isolated and purified UMP kinase may be produced by the recombinant expression systems described above. The method comprises culturing a host cell transformed with an expression vector comprising a DNA sequence that encodes the protein under conditions sufficient to promote expression of the protein. The protein is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. When expression systems that secrete the recombinant protein are employed, the culture medium first may be concentrated. Following the concentration step, the concentrate can be applied to a purification matrix such as a gel filtration medium. Alternatively, an anion exchange resin can be employed, e.g., a matrix or substrate having pendant diethylaminoethyl (DEAE) groups. The matrices can be acrylamide, agarose, dextran, cellulose, or other types commonly employed in protein purification. Also, a cation exchange step can be employed. Suitable cation exchangers include various insoluble matrices comprising sulfopropyl or carboxymethyl groups. Further, one or more reversed-phase high performance liquid chromatography (RP-HPLC) steps employing hydrophobic RP-HPLC media (e.g., silica gel having pendant methyl or other aliphatic groups) can be employed to further purify the protein. Some or all of the foregoing purification steps, in various combinations, are well known in the art and can be employed to provide an isolated and purified recombinant protein. These and other methods are disclosed in Samrook et al, Molecular Cloning: A Laboratory Manual, Cold Spring Harbor Laboratory Press, Cold Spring Harbor, N.Y., 1989.

In the methods of detecting, characterizing or stabilizing a UMP kinase, GTP and UTP are typically used. However, other nucleotides and/or nucleotide analogs may be used in accordance with the disclosure in the present application. Analogs of GTP, UTP, TTP, ATP, and CTP are known in the art.

### EXAMPLES

Chemicals. Nucleotides, restriction enzymes, T4 DNA ligase T7 DNA polymerase and coupling enzymes were from Roche-Diagnostics or from Sigma. NDP kinase from Dictyostelium discoideum (2000 U/mg of protein) was kindly provided by M. Véron.

Bacterial Strains, Plasmids, Growth Conditions and DNA Manipulations. General DNA manipulations were performed as described by Sambrook et al. [Sambrook, 1989 #66]. The pyrH gene from B. subtilis was amplified by polymerase chain reaction using chromosomal DNA from the strain 168 () as the matrix. The product was inserted between the NdeI and XhoI restriction sites of plasmid pET24a (Novagen). The resulting plasmid (pSL13) was introduced into strain BL21(DE3)/pDIA17 () to overproduce the UMP kinase. Recombinant strain was grown in 2YT medium supplemented with antibiotics to an optical density of 1 at 600 nm. Then overproduction was triggered by isopropyl-β-D-thiogalactoside induction (1 mM final concentration) for 3 h, then bacteria were harvested by centrifugation.

Purification of UMP kinase and activity assays. E. coli overproducing the UMP kinase from B. subtilis was disrupted by sonication in 50 mM Tris-HCl (pH 7.4) and 2 mM UTP. The bacterial extract was heated for 10 min at 65° C., then the precipitated proteins were removed by centrifugation at 10,000 g for 30 min. The supernatant was concentrated by ultrafiltration to about 10 mg of protein/ml, then applied to a Sephacryl S-300 HR column (2.5×110 cm) equilibrated with 50 mM Tris-HCl (pH 7.4), 0.1 M NaCl and 2 mM UTP at a flow rate of 10 ml/h. The peak fraction containing over 95% pure UMP kinase was concentrated again to 10 mg of protein/ml. Fractions of 1 ml of protein solution were sampled in Eppendorf tubes and stored at different temperatures between 20° C. and −80° C. The UMP kinase activity was determined at 30° C. using a coupled spectrophotometric assay (0.5 ml final volume) on an Eppendorf PCP6121 photometer [Blondin, 1994 #977]. The “standard” reaction medium contained 50 mM Tris-HCl (pH 7.4), 50 mM KCl, 2 mM MgCl₂, 1 mM phosphoenolpyruvate, 0.2 mM NADH, 2 mM ATP, 0.5 mM GTP and 2 units each of lactate dehydrogenase, pyruvate kinase and NDP kinase. The crude or pure preparation of UMP kinase was then added, followed two minutes later by 1.3 mM UMP. The decrease in absorbance at 334 nm (between 0.03 and 0.3/min) was then recorded and corrected for secondary reactions, occurring in the absence of UMP. One unit of UMP kinase corresponds to 1 μmol of product formed per min.

Immunochemical methods. Anti-UMP kinase sera were obtained by immunizing rabbits with 250 μg of purified recombinant protein at 12 days intervals. After four injections, the rabbits were bled and polyclonal response tested by ELISA. Immune sera were adsorbed against an E. coli sonicate to improve the signal-to-noise ratio. Western blotting was performed after SDS-polyacrylamide gel electrophoresis (SDS-PAGE) by transfer of proteins on a nitrocellulose membrane, followed by treatment with 1:1000 dilution of polyclonal sera and alkaline phosphatase-conjugated anti-rabbit immunoglobulins. Alkaline phosphatase activity was revealed by using the nitroblue tetrazolium-5-bromo-4-chloro-3-indolylphosphate dye system.

Two-dimensional gel electrophoresis. B. subtilis strain 168 was grown in 2YT medium until an optical density of 0.5 at 600 nm, harvested by centrifugation, then sonicated in 50 mM Tris-HCl (pH 8) containing DNAse and RNAse (final concentration of 1 mg/ml and 0.5 mg/ml, respectively). Insoluble material was removed by centrifugation, and supernatants boiled for 5 min with 0.3% SDS and 50 mM dithiothreitol (DTT). Extracts were quickly frozen in liquid nitrogen, lyophilized, then resuspended in 9.95 M urea, 4% NP40, 2% ampholytes, 100 mM DTT, and stored at −20° C. until used. The electrophoresis procedure was previously described [Garrels, 1983 #821; Laurent-Winter, 1997 #824], with some modifications. Samples containing 50 μg protein were loaded onto the isoelectric focusing gel (IEF, Millipore Inc. ampholytes, pH range 3 to 10), focused for 20,000 volt×h, and the second dimension was performed on 10% slab gels. Detection of proteins was performed by silver nitrate staining according to Morrissey [Morrissey, 1981 #827]. Molecular masses, isoelectric points (pI), and spot quantifications were determined using the Melanie II software, and the GS-700 densitometer (Biorad) [Landais, 1999 #1080].

Immunoelectron microscopy. Bacteria were fixed with 4% formaldehyde (freshly made from paraformaldehyde) and 0.2% glutaraldehyde in 0.1 M cacodylate buffer (pH 7.4), for one hour at 4° C. The cell pellets were rinsed with cacodylate buffer, then treated with 0.5% aqueous uranyl acetate solution [Benichou, 1990 #819], followed by a final rinse in distilled water. Bacteria were embedded in 2% agarose (type IX, Sigma). Small blocks were embedded in Unicryl by the PLT method and modified procedure, as described by Gounon and Rolland (15) [Gounon, 1999 #1880]. Ultrathin sections were collected on Formvar-carbon coated nickel grids. Sections were then incubated in the following solutions: PBS containing 50 mM NH₄Cl: 10 min; PBS containing 1% BSA and 1% normal goat serum [Brorson, 1997 #817]: 10 min; rabbit polyclonal anti-UMP kinase antisera (1/100 dilution), or mouse monoclonal anti-CMP kinase antibodies (100 μg/ml): one hour. Two washes (5 min each) were performed in PBS containing 0.1% BSA, then one wash in PBS. Incubations were for 45 min in a solution containing anti-rabbit or anti-mouse gold-conjugated immunoglobulin (5 nm or 10 nm particles, British Biocell Laboratories, Cardiff, UK), diluted 1/20 in PBS containing 0.01% fish skin gelatin (Sigma). Sections were washed once in PBS and three times in distilled water, then fixed for 2 min with 1% glutaraldehyde in 0.1 M cacodylate buffer (pH 7.4), and finally rinsed with distilled water and dried. Optional counterstaining was performed by treating the sections with 2% aqueous uranyl acetate solution for 40 min, followed by a 3 min incubation in Millonig's lead tartrate solution [Millonig, 1961 #826]. Specimens were examined with a Philips CM12 electron microscope operating under standard conditions [Landais, 1999 #1080].

Other analytical procedures. Protein concentration was measured according to Bradford [Bradford, 1976 #68]. Ion spray mass spectra were recorded on a quadrupole mass spectrometer API-365 (Perkin-Elmer) equipped with an ion spray (nebulizer-assistant electrospray) source. The sample (˜2 pmol.μl⁻¹) dissolved in 20 % acetonitrile in water and 0.1% HCOOH was delivered to the source at a flow rate of 5 μl.min⁻¹. SDS-PAGE was performed as described by Laemmli [Laemmli, 1970 #69]. The proteins bands from SDS-PAGE were electroblotted into a Problott membrane filter (Applied Biosystems) and detected by staining in the Coomassie Blue. The N-terminal amino acid sequence of the protein from the excised band was determined by a protein sequencer (Applied Biosystems, Inc.). Fluorescence experiments were performed on a Perkin-Elmer LS-5B luminescence spectrometer thermostated at 25° C. Emission spectra of UMP kinase (λexc=295 nm; band width=5 nm) were recorded from 305 to 400 nm.

### Results

Cloning, sequencing of pyrH gene from B. subtilis and complementation tests in E. coli. The pyrH gene from B. subtilis was cloned by PCR into the expression vector pET24a, and sequenced. The resulting ORF showed two differences when compared with the published databank: one additional T at bp170, and one missing A at bp185. As a consequence, the ORF of the pyrh gene displays a double frame-shift of 14 bp long stretch, resulting in four amino acid residues change: ⁵⁷LeuTrpArgGly⁶⁰ instead of TyrGlyAlaGlu in the original sequence. Harbored on high-copy number vectors, the B. subtilis pyrh gene complemented the thermosensitive phenotype of strain KUR1244 (pyrH88ts) of E. coli indicating that it was functional. Complementation experiments performed on strain MC4100-42-14:40 (car: :lacZpyrH42), in which expression of the car: :lacZ fusion is repressed in the presence of wild-type UMP kinase activity, showed that in high copy-number, the pyrh gene from B. subtilis resulted in a significant repression of β-galactosidase activity.

Stability of recombinant UMP kinase in crude bacterial extracts.

UMP kinase from B. subtilis overproduced in strain BL21 (DE3)/pDIA17 (over 30% of total E. coli proteins) was recovered in the supernatant after cell breakage in 50 mM Tris-HCl (pH 7.4) and centrifugation. The activity of recombinant UMP kinase declined rapidly upon storage at either room temperature, +4° C. or under frozen state. After 24 h, only 15% of initial UMP kinase activity was recovered. EDTA, thiols, bovine serum albumin, or antiprotease compounds alone or in mixture were ineffective. UTP (≧2 mM) stabilized considerably the bacterial UMP kinase when stored at various temperatures. A mixture of ATP and GTP (2 mM each) was equally effective. UTP increased the thermal stability of bacterial UMP kinase (FIG. 1), the half maximal inactivation being shifted from 42° C. in the absence of UTP to over 70° C. in the presence of nucleotide.

Purification and molecular characterization of recombinant UMP kinase from B. subtilis.

UMP kinase from B. subtilis overproduced in strain BL21(DE3)/pDIA17 was purified as described under experimental procedures, i.e. a heating step followed by gel permeation chromatography (FIG. 2). The molecular mass of B. subtilis UMP kinase (26,084.2 ±1.7 Da), measured by ESI-MS was in agreement with that calculated (26,083 Da) from the sequence. Gel permeation chromatography yielded a single symmetrical peak of protein consistent with an oligomeric enzyme (6 subunits/oligomer). Ultracentrifugation analysis by sedimentation equilibrium indicated that the dominant species (156 KDa) corresponded to the hexameric enzyme, eventought oligomers of higher molecular mass were also identified. In contrast to E. coli UMP kinase, the B. subtilis enzyme was sensitive to trypsin digestion. The inactivation of the bacterial enzyme at 30° C. and in a 1/500 (w/w) trypsin/UMP kinase ratio followed a first order kinetics (t_{½}=min). SDS-PAGE analysis of the digested enzyme showed accumulation of stable fragments. The truncated protein was still oligomeric as indicated by gel permeation chromatography. ESI-MS analysis of the partially cleaved protein indicated that the stable fragment (18,905.8±0.5 Da) is a C-terminal truncated form (residues 1 to 174). A genetically engineered N-terminal His-tagged truncated form of enzyme was found essentially instable in the sonicated bacterial extract. The soluble fraction was purified by Ni-NTA chromatography. Gel permeation chromatography and appropriate molecular mass markers (lactate dehydrogenase, 140 KDa; creatine kinase, 82 KDa; and E. coli adenylate kinase, 27 KDa) indicated molecular mass of the recombinant protein of 120 KDa, consistent with a hexameric form. Urea induced denaturation of native and C-terminal truncated form of B. subtilis UMP kinase was monitored from the intrinsec fluorescence of the single Trp residue (W58). As shown in FIG. the native intact UMP kinase irrespective of the presence or the absence of the His-tag exhibits upon excitation at 295 nm a fluorescence emission spectrum with maximum at nm which indicates that W58 is located in a hydrophobic environment, not exposed to the solvent. Urea over xM increased slightly the fluorescence maxima with shift to the red side of the emission spectrum. The mid point transition concentration of urea was xM. Under similar experimental conditions, the C-terminal truncated form of B. subtilis UMP kinase showed a significantly lower stability against denaturation by urea.

Presence of the pyrH gene product in B. subtilis 168 wild-type strain.

To determine the presence of pyrH/smbA gene product in the B. subtilis strain 168, the bacterial extract in 50 mM Tris-HCl (pH 7.4) and 2 mM UTP was heated at 65° C. for 10 min, to inactivate ATPase activity interfering in the spectrophometric assay. The specific activity of the protein in the crude extract under “standard” assay conditions (see Experimental Procedures) was of 0.016 U/mg protein. Since the purified recombinant protein has a specific activity of 26 U/mg of protein under identical experimental conditions, we might assume a protein abundancy in B. subtilis extract between 0.06 and 0.1%, a figure close to that found in E. coli where UMP kinase represents 0.05% of total proteins. The UMP kinase was identified in the 2D-PAGE map of wild-type strain 168 by comigration with recombinant protein. Further unambiguous identification of enzyme was performed by 2D western blotting using rabbit polyclonal antibodies raised against the recombinant protein. The B. subtilis pyrH gene product migrates at pI= and apparent molecular mass of KDa. Densitometric scanning of silver stained 2D gels indicated a protein abundancy of 0.15% which fitted reasonably well with that calculated from specific activities.

Kinetic properties of UMP kinase from B. subtilis.

Determination of UMP kinase activity with various nucleoside triphosphates and UMP at fixed concentrations (1 mM) indicated low specific activities, the maximal rate being with ATP. When NTPs were used in mixture, the highest specific activity was obtained with ATP and GTP, indicating an almost absolute requirement for GTP for expression of full catalytic activity dATP was as good as ATP as phosphate donor. When ATP concentration was varied in the absence or in the presence of GTP (0.5 mM) at a single concentration of UMP (1 mM), the apparent Km for ATP was unusually high (0.9 mM). When ATP was constant (1 mM) the kinetics with variable concentrations of UMP was strongly dependant on GTP. Thus in absence of GTP, the rates were maximal at 50-70 μM UMP (Km for UMP≈8 μM) to decline upon further increase in UMP. In the presence of GTP, the saturation was attained at 0.2 mM UMP. The apparent Km for UTP was 30 μM without inhibition by excess of UMP. GTP showed by far the most important effect, i. e. a ten fold activation with 2 mM ATP and 1 mM UTP, the half maximum activation being reach at 0.1 mM nucleoside triphosphate. dGTP was also effective but with lower affinities and extent of activation whereas GMP was totally ineffective. These results are in contrast with those obtained with E. coli UMP kinase, where GMP, cGMP and even guanosine exerted a significant activation. UTP antagonized the effect of GTP. In the absence, UTP decreased the reaction rate with an I₅₀ value of approximately60 μM. At lower concentration of UMP (50 μM) the I₅₀ of UTP inhibition was decreased to 50 μM.

Effect of uridine nucleotide analogs on UMP kinase from B. subtilis.

Molecular cloning and overexpression of UMP kinase from the three organisms

The pyrH/smbA gene encoding UMP kinase from M. tuberculosis, B. subtilis and H. influenzae was cloned by PCR amplification, using as template DNA isolated from the three strains employed for the whole genome sequencing.

a) The pyrH gene of M. tuberculosis was cloned using the PRO bacterial expression system developed by Clontech. The vector used is a derivative of PROTet were the recombinant protein is fused to an N-terminal 6×HN affinity tag. This system is optimized for use with TALON resins, which are cobalt-based IMAC (immobilized metal ion affinity chromatography) resins. UMP kinase expressed in DH5αPRO E. coli strain was induced when bacteria reached an OD of 0.7 by addition of anhydrotetracycline (final concentration of 100 μg/liter). Bacteria were collected after incubation at 37° C. for 16 hours. The plasmid containing the pyrH gene from M. tuberculosis has been deposited at the CNCM on Aug. 8, 2000 under the accession number 1-2542.

b) The pyrh gene from B. subtilis and H. influenzae was inserted into the plasmid pET22b (Novagen). The resulting plasmids are under the control of a hybrid promoter/operator region constituted from the T7 promoter followed by the lac operator. They were introduced into the strain BL21 (DE3) of E. coli (Novagen) that is producing the T7RNA polymerase. The strains were grown in LB medium supplemented with kanamycin (30 mg/liter) and chloramphenicol (30 mg/liter). Induction was made with 1 mM IPTG. Three hours after induction bacteria were collected by centrifugation and stored at −20° C. until use. The plasmid containing the pyrH gene from B. subtilis has been deposited at the CNCM on Nov. 17, 2000 under the accession number 1-2579. The plasmid containing the pyrH gene from H. influenzae smbA insert has been deposited at the CNCM on Oct. 26, 2000 under the accession number I-2574.

Purification and storage of UMP kinase from the three organisms

a) The recombinant UMP kinase from M. tuberculosis was purified according to the CLONTECH's TALON Purification Kit. The protein was eluted by increasing imidazole concentration from 5 to 150 mM. The tagged UMP kinase exhibits the same properties as the wild-type UMP kinase (hexamer, activation by GTP, inhibition by UTP Tm of 67° C. in the presence of UTP and 52° C. in the absence of UTP). It is soluble at >10 mg/ml in 50 mM phosphate buffer (pH 7.4) with 100 mM NaCl and stable at 4° C. for at least three months.

b) The recombinant UMP kinases from B. subtilis and H. influenzae were purified using essentially the protocol described below. The bacteria were disrupted by sonication in 50 mM Tris-HCl pH 7.4 and 2 mM UTP. The bacterial extract was heated for 10 min at 65° C. (B. subtilis) or 70° C. (H. influenzae) then the precipitated proteins were removed by centrifugation at 1000 g for 30 min. The supernatant was concentrated by ultrafiltration then applied to a Sephacryl S-300 HR column (2.5×110 cm) equilibrated with 50 mM Tris-HCl pH 7.4, 0.1 mM NaCl, and 2 mM UTP at a flow rate of 10 ml/h. The peak fractions containing over 95% pure UMP kinase were pooled and concentrated again to 10-15 mg of protein/ml. Protein solution was kept at −20° C.

c) Important notice for storage

Whereas UMP kinase from M. tuberculosis is stable in the absence of UTP the enzyme from the other two organisms (particularly that from B. subtilis) are unstable in the absence of the nucleotide, irrespective of the storage temperature (+4° C., −20° C. or −80° C.).

Activity assay

The UMP kinase activity was determined at 30° C. using a coupled spectrophotometric assay (0.5 ml final volume) on an Eppendorf PCP6121 photometer. The “standard” reaction medium contained 50 mM Tris-HCl (pH 7.4), 50 mM KCl, 2 mM MgCl₂, 1 mM phosphoenolpyruvate, 0.2 mM NADH, 1 or 2 mM ATP, 0.5 mM GTP and 2 units each of lactate dehydrogenase, pyruvate kinase and NDP kinase. The pure preparation of UMP kinase was then added, followed by 0.3 or 1 mM UMP. The decrease in absorbance at 334 nm (between 0.03 and 0.3/min) was then recorded and corrected for secondary reactions occurring in the absence of UMP. One unit of UMP kinase corresponds to 1 μmol of product formed per min. The specific activities of the recombinant enzymes are the following:

30 units/mg of protein for UMP kinase from M. tuberculosis (1 mM ATP, 0.3 mM UMP).

25 units/mg of protein for UMP kinase from B. subtilis (2 mM ATP, 1 mM UMP).

45 units/mg of protein for UMP kinase from H. influenzae (1 mM ATP, 1 mM UMP).

It should be noticed that the degree of activation by GTP is variable (a factor of 2 for UMP kinase from M. tuberculosis, of 3 for H. influenzae, and between 10 and 20 for B. subtilis). There are also differences concerning the best activator: in the case of H. influenzae the highest activation and affinity for UMP kinase is shown by cGMP, maximal activation being attained at 20 μM nucleotide.

Other UMP kinases

Continuing our searches for soluble and stable UMP kinases we identified two other enzymes not yet fully characterized: Streptococcus pneumoniae (gram positive) and Neisseria meningiditis (gram negative). The enzyme from the first organism resembles that from B. subtilis (high factor of activation by GTP), the enzyme from the second resembles that of E. coli and H. influenzae.

The ORFs of the pyrH gene from the three organisms

They are available in the gene databank. It should be mentioned, however, that we found two differences with the published sequence of B. subtilis pyrH gene: one additional T at bp 170, and one missing A at bp185. As a consequence, the ORF of the pyrH gene displays a double frame-shift of 14 bp long stretch, resulting in four amino acid residues change: ⁵⁷LeuTrpArgGly⁶⁰ instead of TyrGlyAlaGlu in the original deduced protein sequence. The B. subtilis pyrH gene is shown SEQ ID NO: 1, the sequence of the H. influenzae pyrH gene is shown in SEQ ID NO:2, and the sequence of the M. tuberculosis gene is shown in SEQ ID NO:3.

All patents and publications mentioned herein are incorporated herein by reference to the extent allowed by law for the purpose of describing and disclosing the proteins, enzymes, vectors, host cells, and methodologies reported therein that might be used with the present invention.

References

1. L. Serina, C. Blondin, E. Krin, O. Sismeiro, A. Danchin, H. Sakamoto, A. -M. Gilles & O. Barzu. (1995). Escherichia coli UMP-kinase, a member of the aspartofinase family, is a hexamer regulated by guanine nucleotides and UTP. Biochemistry, 34, 5066-5074/

2. L. Serina, N. Bucurenci, A. -M. Gilles, W. K. Surewicz, H. Fabian, H. H. Mantsch, M. Takahashi, I. Petrescu, G. Batelier & O. Bârzu. (1996). Structural properties of UMP kinase from Escherichia coli: modulation of protein solubility by pH and UTP. Biochemistry, 35, 7003-7011.

3. N. Bucurenci, L. Serina, C. Zaharia, S. Landais, A. Danchin & O. Bârzu. (1998). Mutational analysis of UMP kinase from Escherichia coli. J. Bacteriol., 180, 473-477.

4. S. Landais, P. Gounon, C. Laurent-Winter, J. -C. Mazie, A. Danchin, O. Bârzu & H. Sakamoto. (1999). Immunochemical analysis of UMP kinase from Escherichia coli. J. Bacteriol. 181, 833-840.

5. O. Bârzu, A. -M. Gilles & P. Briozzo. (1999). Bacterial UMP-and CMP kinases are distinct and structurally unrelated entities. Paths to Pyrimidines 7, 86-95.

1. An isolated polynucleotide comprising a sequence selected from the group consisting of SEQ ID NO:1, SEQ ID NO:2, and SEQ ID NO:3, wherein said polynucleotide encodes a protein having UMP kinase activity.

2. An isolated polynucleotide which hybridizes to the polynucleotide of claim 1 and which encodes a protein having UMP kinase activity.

3. An expression vector comprising the polynucleotide of claim 1.

4. A host cell which is transformed with the expression vector of claim 3.

5. A method of purifying a UMP kinase comprising;
culturing the host cell of claim 4 under conditions suitable for expressing UMP kinase encoded by said polynucleotide; and
collecting said UMP kinase.

6. The method of claim 5, wherein said collecting comprises adding UTP to said UMP kinase.

7. The method of claim 5 further comprising purifying said UMP kinase.

8. The method of claim 7, wherein said purifying comprises adding UTP to said UMP kinase.

9. A method of detecting the presence of a UMP kinase in a sample comprising
adding GTP to said sample; and
assaying for UMP kinase activity, wherein an increase in UMP kinase activity correlates to the presence of UMP kinase.

10. A method of detecting the presence of a UMP kinase in a sample comprising
adding UTP to said sample; and
assaying for UMP kinase activity, wherein a decrease in UMP kinase activity correlates to the presence of UMP kinase.

11. A method of detecting the presence of a UMP kinase in a sample comprising
adding GTP to said sample;
assaying for UMP kinase activity, wherein an increase in UMP kinase activity correlates to the presence of UMP kinase;
adding UTP to said sample; and
assaying for UMP kinase activity, wherein a decrease in UMP kinase activity correlates to the presence of UMP kinase.

12. A method of stabilizing a UMP kinase in a sample, comprising adding UTP to said sample.

13. A method of activating a UMP kinase in a sample, comprising adding GTP to said sample.

14. The method of claim 12, further comprising adding GTP to said sample.

15. A method of screening for molecules according to any one of claims 5 to 14.

16. An active molecule as obtained according to the method of any one of claims 5 to 14.

17. An active molecule as obtained according to the method of screening of claim 15.

18. The recombinant bacteria strains deposited at the CNCM under the accession numbers I-2542, I12574 and I12579.
